(19) United States
(12) Patent Application Publication
(10) Pub. No.: US 2012/0239020 A1
(43) Pub. Date: Sep. 20, 2012

(54) Geo-Location Addition to Electrosurgical Generator

(75) Inventor: James S. Cunningham, Boulder, CO (US)

(73) Assignee: TYCO Healthcare Group LP, Boulder, CO (US)

(21) Appl. No.: 13/048,639

(22) Filed: Mar. 15, 2011

(51) Int. Cl. A61B 18/12; A61B 18/18

(52) U.S. Cl. 606/33; 606/34

(57) ABSTRACT

A system and method for determining the location of an electrosurgical generator using a geo-location device within the generator. The geo-location device determines the location of the generator and the controller sets a default language of the generator based on the determined location. The default language may be overridden by a user when necessary. The geo-location device is coupled to a communication port. The communication port allows for a wireless signal to be sent upon the generator being reported stolen or for tracking location of the generators. The communication port is coupled to the controller to allow for remote disablement, for example in response to the generator being stolen. Alternatively, the controller may disable the generator when the geo-location device determines that the generator has moved outside a predetermined location.

### BACKGROUND

1. Technical Field

The present disclosure relates to electrosurgical generators. More particularly, the present disclosure relates to a system and method for determining a location of an electrosurgical generator.

2. Background of Related Art

Energy-based tissue treatment is well known in the art. Various types of energy (e.g., electrical, ultrasonic, microwave, cryogenic, heat, laser, etc.) are applied to tissue to achieve a desired result. Electrosurgery involves application of high radio frequency electrical current to a surgical site to cut, ablate, coagulate or seal tissue.

In bipolar electrosurgery, one of the electrodes of the hand-held instrument functions as the active electrode and the other as the return electrode. The return electrode is placed in close proximity to the active electrode such that an electrical circuit is formed between the two electrodes (e.g., electrosurgical forceps). In this manner, the applied electrical current is limited to the body tissue positioned between the electrodes. When the electrodes are sufficiently separated from one another, the electrical circuit is open and thus inadvertent contact with body tissue with either of the separated electrodes does not cause current to flow.

Bipolar electrosurgical techniques and instruments can be used to coagulate blood vessels or tissue, e.g., soft tissue structures, such as lung, brain and intestine. A surgeon can either cauterize, coagulate/desiccate and/or simply reduce or slow bleeding, by controlling the intensity, frequency and duration of the electrosurgical energy applied between the electrodes and through the tissue. In order to achieve one of these desired surgical effects without causing unwanted charring of tissue at the surgical site or causing collateral damage to adjacent tissue, e.g., thermal spread, it is necessary to control the output from the electrosurgical generator, e.g., power, waveform, voltage, current, pulse rate, etc.

In monopolar electrosurgery, the active electrode is typically a part of the surgical instrument held by the surgeon that is applied to the tissue to be treated. A patient return electrode is placed remotely from the active electrode to carry the current back to the generator and safely disperse current applied by the active electrode. The return electrodes usually have a large patient contact surface area to minimize heating at that site. Heating is caused by high current densities that directly depend on the surface area. A larger surface contact area results in lower localized heat intensity. Return electrodes are typically sized based on assumptions of the maximum current utilized during a particular surgical procedure and the duty cycle (i.e., the percentage of time the generator is on).

The electrosurgical generator incorporates software and firmware for monitoring and control. One of the features of the software is a language setting where a user can choose from over twenty five languages. However, selecting a language through menus may be cumbersome or the language selected may be inadvertently changed by a user.

### SUMMARY

In accordance with the present disclosure, a system and method for determining the location of an electrosurgical generator using a geo-location device within the generator. The geo-location device determines the location of the generator and the controller sets a default language of the generator based on the determined location. The default language may be overridden by a user when necessary. The geo-location device is coupled to a communication port. The communication port allows for a wireless signal to be sent upon the generator being reported stolen or for tracking location of the generators. The communication port is coupled to the controller to allow for remote disablement, for example in response to the generator being stolen. Alternatively, the controller may disable the generator when the geo-location device determines that the generator has moved outside a predetermined location.

According to an embodiment of the present disclosure, a method for operating an electrosurgical generator includes the steps of connecting a geo-location device to a controller within the generator and determining a location of the generator. The method further includes the steps of automatically selecting a default language based on the determined location, and modifying a display screen based on the default language.

According to another embodiment of the present disclosure, an electrosurgical generator includes a power supply and a RF output state configured to generate an electrosurgical waveform. The generator further includes a geo-location device configured to determine a location of the electrosurgical generator and a controller coupled to the geo-location device. The controller configured to automatically set a default language based on the location determined by the geo-location device.

According to another embodiment of the present disclosure, a method of operating an electrosurgical generator includes the steps of installing a geo-location device within the generator, and mapping the geo-location device to a generator ID of the generator. The method further includes the steps of determining a location of the generator, and sending, wirelessly, the location of the generator to a remote device.

### BRIEF DESCRIPTION OF THE DRAWINGS

Various embodiments of the present disclosure are described herein with reference to the drawings wherein:

FIG. 1 is a schematic diagram of an electrosurgical system according to one embodiment of the present disclosure;

FIG. 2 is a front view of an electrosurgical generator according to an embodiment of the present disclosure;

FIG. 3 is a schematic block diagram of the electrosurgical generator of FIG. 2 according to an embodiment of the present disclosure; and

FIG. 4 is a flow chart of a method according to an embodiment of the present disclosure.

### DETAILED DESCRIPTION

Particular embodiments of the present disclosure are described hereinbelow with reference to the accompanying drawings. In the following description, well-known functions or constructions are not described in detail to avoid obscuring the present disclosure in unnecessary detail.

The generator according to the present disclosure can perform monopolar and bipolar electrosurgical procedures, including vessel sealing procedures. The generator may include a plurality of outputs for interfacing with various electrosurgical instruments (e.g., a monopolar active electrode, return electrode, bipolar electrosurgical forceps, footswitch, etc.). Further, the generator includes electronic circuitry configured to generate radio frequency power specifically suited for various electrosurgical modes (e.g., cutting, blending, division, etc.) and procedures (e.g., monopolar, bipolar, vessel sealing).

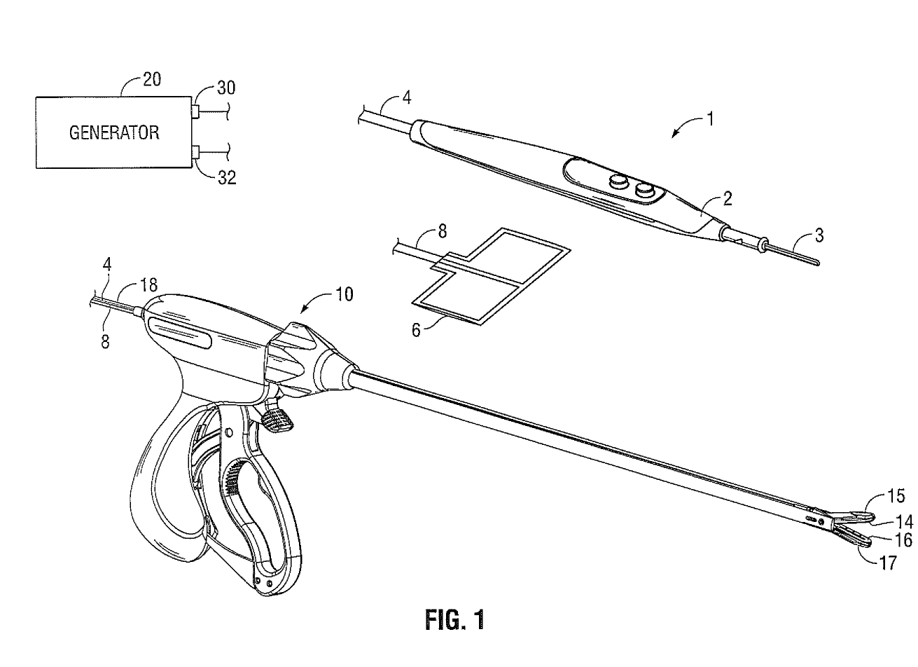

FIG. 1 is a schematic illustration of a bipolar and monopolar electrosurgical system 1 according to one embodiment of the present disclosure. The system 1 includes one or more monopolar electrosurgical instruments 2 having one or more electrodes 3 (e.g., electrosurgical cutting probe, ablation electrode(s), etc.) for treating tissue of a patient. Electrosurgical RF energy is supplied to the instrument 2 by a generator 20. The instrument 2 includes an active electrode 3 that is connected via a supply line 4 to an active terminal 30 of the generator 20, allowing the instrument 2 to coagulate, ablate and/or otherwise treat tissue. The energy is returned to the generator 20 through a return electrode 6 via a return line 8 at a return terminal 32 of the generator 20. The system 1 may include a plurality of return electrodes 6 that are arranged to minimize the chances of tissue damage by maximizing the overall contact area with the patient. In addition, the generator 20 and the return electrode 6 may be configured for monitoring so-called “tissue-to-patient” contact to insure that sufficient contact exists therebetween to further minimize chances of tissue damage.

The system 1 may also include a bipolar electrosurgical forceps 10 having one or more electrodes for treating tissue of a patient. The electrosurgical forceps 10 includes opposing jaw members 15 and 17 having one or more active electrodes 14 and a return electrode 16 disposed therein, respectively. The active electrode 14 and the return electrode 16 are connected to the generator 20 through cable 18 that includes the supply and return lines 4, 8 coupled to the active and return terminals 30, 32, respectively. The electrosurgical forceps 10 is coupled to the generator 20 at a connector having connections to the active and return terminals 30 and 32 (e.g., pins) via a plug disposed at the end of the cable 18, wherein the plug includes contacts from the supply and return lines 4, 8.

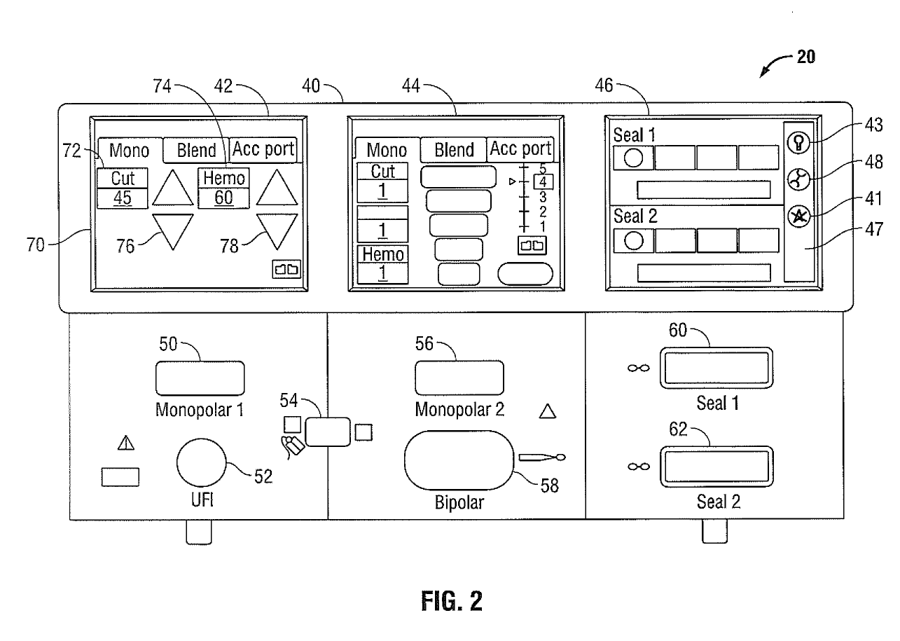

With reference to FIG. 2, front face 40 of the generator 20 is shown. The generator 20 may be any suitable type (e.g., electrosurgical, microwave, etc.) and may include a plurality of connectors 50-62 to accommodate various types of electrosurgical instruments (e.g., multiple instruments 2, electrosurgical forceps 10, etc.). The generator 20 includes one or more display screens 42, 44, 46 for providing the user with a variety of output information (e.g., intensity settings, treatment complete indicators, etc.). Each of the screens 42, 44, 46 is associated with a corresponding connector 50-62. The generator 20 includes suitable input controls (e.g., buttons, activators, switches, touch screen, etc.) for controlling the generator 20. The display screens 42, 44, 46 are also configured as touch screens that display a corresponding menu for the electrosurgical instruments (e.g., multiple instruments 2, electrosurgical forceps 10, etc.). The user then makes inputs by simply touching corresponding menu options. The controls allow the user to select desired output modes as well as adjust operating parameters of the modes, such as power, waveform parameters, etc. to achieve the desired output suitable for a particular task (e.g., cutting, coagulating, tissue sealing, etc.). Additionally, the user can override a default setting for language by touching corresponding menu options.

The generator 20 is configured to operate in a variety of modes. In one embodiment, the generator 20 may output the following modes, cut, blend, division with hemostasis, fulgurate and spray. Each of the modes operates based on a preprogrammed power curve that dictates how much power is outputted by the generator 20 at varying impedance ranges of the load (e.g., tissue). Each of the power curves includes a constant power, constant voltage and constant current ranges that are defined by the user-selected power setting and the measured minimum impedance of the load.

In the cut mode, for example, the generator 20 supplies a continuous sine wave at a predetermined frequency (e.g., 472 kHz) having a crest factor of 1.5 or less in the impedance range of 100Ω to 2,000Ω. The cut mode power curve may include three regions: constant current into low impedance, constant power into medium impedance and constant voltage into high impedance. In the blend mode, the generator supplies bursts of a sine wave at the predetermined frequency, with the bursts reoccurring at a first predetermined rate (e.g., about 26.21 kHz). In one embodiment, the duty cycle of the bursts may be about 50%. The crest factor of one period of the sine wave may be less than 1.5. The crest factor of the burst may be about 2.7.

The division with hemostasis mode includes bursts of sine waves at a predetermined frequency (e.g., 472 kHz) reoccurring at a second predetermined rate (e.g., about 28.3 kHz). The duty cycle of the bursts may be 25%. The crest factor of one burst may be 4.3 across an impedance range of 100Ω to 2,000Ω. The fulgurate mode includes bursts of sine waves at a predetermined frequency (e.g., 472 kHz) reoccurring at a third predetermined rate (e.g., about 30.66 kHz). The duty cycle of the bursts may be 6.5% and the crest factor of one burst may be 5.55 across an impedance range of 100Ω to 2,000Ω. The spray mode may be bursts of sine wave at a predetermined frequency (e.g., 472 kHz) reoccurring at a third predetermined rate (e.g., about 21.7 kHz). The duty cycle of the bursts may be 4.6% and the crest factor of one burst may be 6.6 across the impedance range of 100Ω to 2,000Ω.

The screen 46 controls bipolar sealing procedures performed by the forceps 10 that may be plugged into the connectors 60 and 62. The generator 20 outputs energy through the connectors 60 and 62 suitable for sealing tissue grasped by the forceps 10. The screen 46 also controls a system tray 47 to allow the user to access and adjust system settings. The system tray 47 may include a brightness icon 43, a menu icon 48, an error disabled icon 41. The brightness icon 43 allows the user to adjust the brightness of the screens 42, 44, 46. The error disabled icon 41 indicates that the error warnings have been disabled using the service menu. The menu icon 48 allows access to the main menu where the user can change options for language, appearance, and other operations.

The screen 42 controls monopolar output and the devices connected to the connectors 50 and 52. The connector 50 is configured to couple to the instrument 2 and the connector 52 is configured to couple to a foot switch (not shown). The foot switch provides for additional inputs (e.g., replicating inputs of the generator 20 and/or instrument 2). For example, in standard monoploar mode, the power output modes 72, 74 are indicted on interface 70. The user adjusts the power controls using up and down arrows 76, 78 for each mode respectively.

The screen 44 controls monopolar and bipolar output and the devices connected to the connectors 56 and 58. Connector 56 is configured to couple to the instrument 2, allowing the generator 20 to power multiple instruments 2. Connector 58 is configured to couple to a bipolar instrument (not shown). When using the generator 20 in monopolar mode (e.g., with instruments 2), the return electrode 6 is coupled to the connector 54, which is associated with the screens 42 and 44. The generator 20 is configured to output the modes discussed above through the connectors 50, 56, 58.

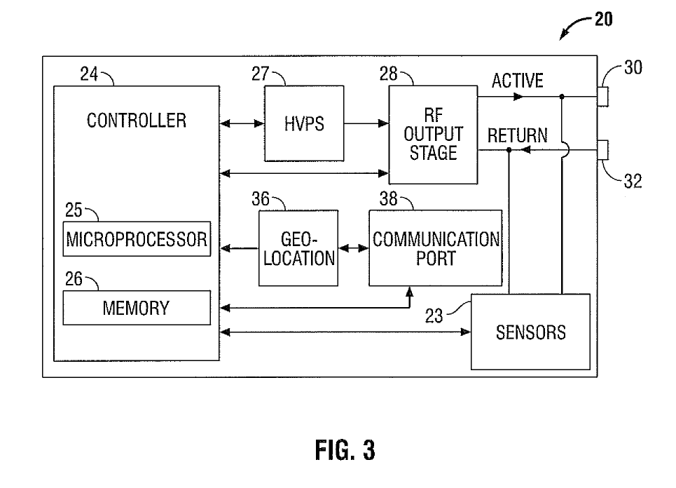

FIG. 3 shows a schematic block diagram of the generator 20 having a controller 24, a high voltage DC power supply 27 (“HVPS”) and an RF output stage 28, a geo-location chip 36, and a communication port 38. The HVPS 27 is connected to an AC source (e.g., electrical wall outlet) and provides high voltage DC power to an RE output stage 28, which then converts high voltage DC power into RF energy and delivers the RF energy to the active terminal 30. The energy is returned thereto via the return terminal 32. In particular, the RF output stage 28 generates sinusoidal waveforms of high RF energy. The RF output stage 28 is configured to operate in a plurality of modes, during which the generator 20 outputs corresponding waveforms having specific duty cycles, peak voltages, crest factors, etc. In another embodiment, the generator 20 may be based on other types of suitable power supply topologies.

The controller 24 includes a microprocessor 25 operably connected to a memory 26, which may be volatile type memory (e.g., RAM) and/or non-volatile type memory (e.g., flash media, disk media, etc.). The microprocessor 25 includes an output port that is operably connected to the HVPS 27 and/or RF output stage 28 allowing the microprocessor 25 to control the output of the generator 20 according to either open and/or closed control loop schemes. Those skilled in the art will appreciate that the microprocessor 25 may be substituted by any logic processor (e.g., control circuit) adapted to perform the calculations discussed herein.

A closed loop control scheme is a feedback control loop, in which one or more sensors 23 measure a variety of tissue and/or energy properties (e.g., tissue impedance, tissue temperature, output current and/or voltage, etc.), and provide feedback to the controller 24. Such sensors may include voltage and current sensors that are coupled to the output terminals 30 and 32 of the generator 20, which are within the purview of those skilled in the art. In response to the sensor signals, the controller 24 controls the HVPS 27 and/or RF output stage 28, which then adjusts the DC and/or RF power supply, respectively. The controller 24 also receives input signals from the input controls of the generator 20, the instrument 2 or forceps 10. The controller 24 utilizes the input signals to adjust power outputted by the generator 20 and/or performs other control functions thereon.

The memory 26 includes software for operating the generator 20. The software includes a choice of over twenty five languages. The geo-location chip 36 determines the location of the generator 20 anywhere in the world. The location given by the geo-location ship 36 may be a country, state, region, address, and/or coordinates. The geo-location chip 36 passes the information to the microprocessor 25 and the microprocessor 25 determines the appropriate default language based on the location determined by the geo-location chip 36.

The geo-location chip 36 may also be connected to a communication port 38. The communication port 38 provides wired and/or wireless communication with an external device (not shown), such as an inventory control system or a theft monitoring system. The communication port 38 may provide remote access to the controller 24 from the external device to remotely disable the generator 20. For example, if the generator 20 is reported stolen, then a theft monitoring system may remotely access controller 24 through communication port 38 and disable the generator 20. In another example, during a clinical trial, the generator 20 may be programmed to stay within set boundaries and may automatically be disabled upon the geo-location chip 36 and the controller 24 determines the location is outside the set boundaries. Additionally, the communication port 38 may be used to track the location of the generator 20 by a remote user accessing the generator 20 through the communication port 38 and reading data from the geo-location chip 36. Alternatively, the communication port 38 may be accessed to remotely update or repair the generator 20.

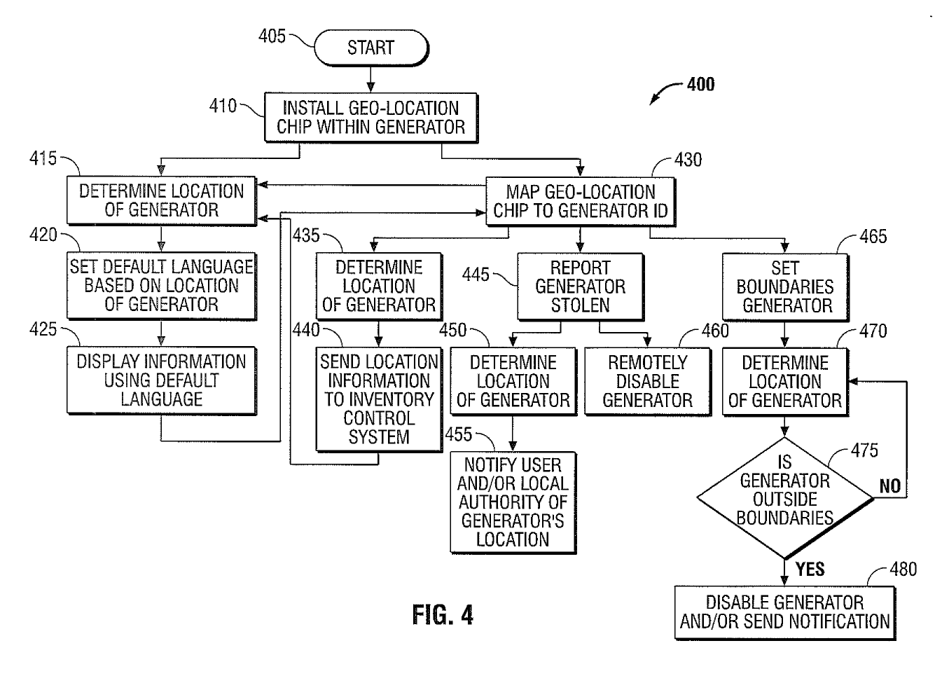

FIG. 4 illustrates a flow diagram 400 for using a geo-location chip 36 within a generator 20. The process 400 starts at step 405, when a geo-location chip 36 is installed within a generator 20. The geo-location chip 36 is connected to controller 24 and communication port 38. The go-location chip 36 determines the location of the generator 20 at step 415. The location may be a country, state, region, address, and/or coordinates of the generator 20. The controller 24 then at step 420 sets the default language of the generator 20 based on the location determined by the geo-location chip 36. The controller adjusts screens 42, 44, 46 to display the default language at step 425. If a user chooses to change the language displayed from the geo-location set default language, the user selects the menu icon 48 on the system tray 47 and picks a different language from a menu.

Next, at step 430, the GPS chip 36 is mapped to a generator ID in a database. The generator ID may be the serial number of the generator 20. The database may be operated and controlled by the manufacturer, a hospital, or other group. Step 430 may take place prior to step 415 and/or after step 425.

For inventory control, the location of the generator 20 is determined by the geo-location chip 36 at step 435. The location is then sent to an inventory control system at step 440 to monitor the location of each generator 20. The location of the generator 20 may be in a warehouse or while shipping. Then, when the generator 20 is turned on for the first time, the generator 20 can set a default language using steps 415-425.

In response to a stolen generator 20, a user may report the generator 20 stolen to the manufacturer of the generator, the hospital, and/or a local authority that may remotely access data from the geo-location chip 36 at step 445. The geo-location chip 36 determines the location of the generator 20 at step 450. The location determined by the geo-location chip 36 is sent to the manufacturer, hospital, and/or local authority using communication port 38 at step 455. Alternatively or in combination with steps 450-455, the manufacturer, hospital, and/or local authority may remotely disable the generator 20 using the communication port 38 at step 460.

In some situations, there may be a need for the generator 20 to be limited to a certain location, such as in a clinical trial or an area with theft problems. Predetermined boundaries for the generator 20 are stored within the memory 26 of the controller 24 at step 465. Next, the geo-location chip 36 determines the location of the generator 20 at step 470. The geo-location chip 36 may check the location periodically, such as once a minute, hour, or day. The controller 24 then determines if the generator 20 is located outside the predetermined boundaries at step 475. If the generator 20 is not outside the location limitations, then the geo-location chip 36 determines the location of the generator 20 again at step 470. If the generator 20 is outside the location limitations, then the generator may be automatically disabled at step 480. Alternatively, a user may be notified of the generator's location and the user may remotely disable the generator 20.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A method for operating an electrosurgical generator, the method comprising:
connecting a geo-location device to a controller within the generator;
determining a location of the generator;
automatically selecting a default language based on the determined location; and
modifying a display screen based on the default language.

2. The method according to claim 1, further comprising:
mapping the geo-location device to a generator ID.

3. The method according to claim 2, further comprising:
sending the location of the generator to an inventory control system.

4. The method according to claim 2, further comprising:
reporting the generator stolen; and
notifying a user of the location of the generator.

5. The method according to claim 4, further comprising:
remotely disabling the generator.

6. The method according to claim 2, further comprising:
programming predetermined boundaries to limit the location of the generator;
determining the generator is outside the boundary limitations; and
in response to determining the generator is outside the boundary limitations, automatically disabling the generator.

7. The method according to claim 2, wherein the generator ID is a serial number of the generator.

8. The method according to claim 1, further comprising;
selecting a different language through a menu to change the language from the default geo-location set language.

9. The method according to claim 1, wherein the location is a state, country, region, address, or coordinates.

10. An electrosurgical generator, comprising:
a power supply;
a RF output state configured to generate an electrosurgical waveform;
a geo-location device configured to determine a location of the electrosurgical generator;
a controller coupled to the geo-location device and configured to automatically set a default language based on the location determined by the geo-location device.

11. The electrosurgical generator according to claim 10, further comprising:
a communication port connected to the geo-location device and the controller.

12. The electrosurgical generator according to claim 11, wherein the communication port is configured to wirelessly send the location of the generator to an external device or user.

13. The electrosurgical generator according to claim 11, wherein the communication port is configured to allow a user remote access to determine location of the generator.

14. The electrosurgical generator according to claim 11, wherein the communication port is configured to receive a software or firmware update.

15. The electrosurgical generator according to claim 11, wherein the communication port is configured to allow a user to remotely repair the generator.

16. The electrosurgical generator according to claim 11, wherein the communication port is configured to allow a user to remotely disable the generator.

17. The electrosurgical generator according to claim 11, further comprising an external database configured to map the geo-location device and a generator ID.

18. A method of operating an electrosurgical generator, the method comprising:
installing a geo-location device within the generator;
mapping the geo-location device to a generator ID of the generator;
determining a location of the generator; and
sending, wirelessly, the location of the generator to a remote device.

19. The method according to claim 18, further comprising:
automatically setting a default language based on the determined location.

20. The method according to claim 18, further comprising:
remotely disabling or updating the generator.
